(19) United States
(12) Patent Application Publication
(10) Pub. No.: US 2007/0239264 A1
(43) Pub. Date: Oct. 11, 2007

(54) Support Prosthesis

(76) Inventor: Thilo Fliedner, Muenchen (DE)

Correspondence Address: ST. ONGE STEWARD JOHNSTON & REENS, LLC, 986 BEDFORD STREET, STAMFORD, CT, 06905-5619, US

(21) Appl. No.: 11/687,499

(22) Filed: Mar. 16, 2007

Related U.S. Application Data
(63) Continuation of application No. PCT/DE05/01633, filed on Sep. 16, 2005.

(30) Foreign Application Priority Data
Sep. 17, 2004 (DE) 10 2004 045 224.5

(51) Int. Cl. A61F 2/88; A61F 2/90

(52) U.S. Cl. 623/1.16; 623/1.22

(57) ABSTRACT

A support prosthesis for vessels or intracorporeal lumens has a large number of support rings which are connected in a longitudinal direction using non-metallic connecting elements.

### CROSS-REFERENCE TO RELATED APPLICATIONS

The present application is a continuation of pending International patent application PCT/DE2005/001633 filed on Sep. 16, 2005 which designates the United States and claims priority from German patent application 10 2004 045 224.5 filed on Sep. 17, 2004, the content of which is incorporated herein by reference.

### FIELD OF THE INVENTION

The invention relates to a support prosthesis for vessels or intracorporeal lumens with a tubular casing which can expand in the radial direction and has a metallic support structure with support elements extending transversely to the longitudinal direction.

### BACKGROUND OF THE INVENTION

A support prosthesis of this type is known from DE 101 53 340 A1. The known support prosthesis has a large number of support rings connected by intermediate rings which are expandable in the longitudinal direction. Both the support rings and the intermediate rings have a meandering course. Support prostheses of this type are also known to a person skilled in the art as stents. The known support prosthesis is used, in particular, for the treatment of vasoconstrictions or what are known as stenoses.

The known support prosthesis is suitable, in particular, for implantation in markedly curved vessels, as the known support prosthesis adapts to the curvature of the vessel to be supported even after expansion. In addition, the known support prosthesis is sufficiently flexible to be able to follow a movement of the vessel, for example a movement of a coronary vessel.

A drawback of the known support prosthesis is that the production of the support rings and the intermediate rings is very complex. Both the support rings and the intermediate rings can in principle be bent from thin wires. In the case of stent diameters within the range of one millimetre, this is possible only with a disproportionately high degree of effort. In addition, it is possible to machine the support rings and intermediate rings from raw material, for example using lasers, although a manufacturing method of this type is cost-intensive and tedious.

Also known from EP 0 997 115 A2 are stents used, in particular, for the treatment of expanded vessels or what are known as aneurysms. For introducing the stents, the diameter of the known stents can be reduced by folding the stents. The known stent can have a large number of rings or a helix which are attached to a tube made of a non-metallic material.

Also known from EP 0 918 496 B1 is a stent likewise used for the treatment of aneurysms. In the known stent, a tubular insert, expandable in the radial direction, is secured by retaining rings which are connected to one another by connecting elements which allow axial relative movement of the retaining rings with respect to one another. Materials proposed for the connecting elements include, inter alia, a polymer material.

Starting from this prior art, the object of the invention is therefore to provide a support prosthesis which can be manufactured easily for the treatment of constrictions.

### SUMMARY OF THE INVENTION

This object is achieved by a support prosthesis having the features of the independent claim. Claims dependent thereon recite advantageous embodiments and developments.

The support prosthesis for vessels or intracorporeal lumens has a tubular casing comprising a metallic support structure with support elements extending transversely to the longitudinal direction. The support elements, which are arranged next to one another in the longitudinal direction, are connected to the tubular casing by at least one connecting element which is made of a resilient, non-metallic material and extends in the longitudinal direction. The connecting elements also allow the support elements to stretch on radial expansion and set the support elements apart from one another on radial expansion.

Non-metallic materials can have such a high modulus of elasticity that the connecting elements do not have to be configured as spring elements by specific shaping. On the contrary, coarse structures can also be selected for the connecting elements. Coarse structures of this type are easy to produce and are able to set the support elements apart from one another on radial expansion. In general, the non-metallic connecting elements are fastened to the support elements by adhesion. The points of contact between the non-metallic connecting elements and the support elements are in this case natural predetermined breaking points, so the connecting elements are at least partially detached from the support elements on radial expansion and can release the support elements for the stretching movement. However, even if there is a positive locking connection between the connecting elements and support elements, the support elements can be pulled out from sufficiently soft connecting elements and therefore become detached from the connecting elements. Despite the coarse configuration of the connecting elements, the stretching movement of the support elements is therefore not be impeded.

In addition, non-metallic materials can have sufficient resilience to allow bending of the support prosthesis in curved vessels and to follow a large number of bending processes of the vessel without breaking. Non-metallic materials can also be biodegradable and dissolve in a patient's body. It is also conceivable to add to the non-metallic material medicaments which are issued to the vessel wall after the introduction of the support prosthesis. Medicaments of this type can, in particular, serve to inhibit inflammatory reactions.

In a preferred embodiment, the support elements have curves which can stretch about a radial axis on radial expansion and the connecting elements are attached in the region of the curves. In this embodiment, the connecting elements can become detached from the support structure, when the curves stretch, in the region surrounding the curves, so the stretching movement is not impeded. In the region of the curves, on the other hand, the connecting elements remain connected to the support elements and set the support elements apart from one another.

The support structure itself can be of differing construction. In one embodiment of the support prosthesis, the support structure comprises support rings which are arranged next to one another, are each expandable in the radial direction and are connected to the casing by the non-metallic connecting elements. In such an embodiment of the support structure, particularly high forces can be exerted onto the wall of the vessel.

In a further embodiment, the support structure is free from support elements which encircle the circumference of the casing in a closed manner. This prevents any extensive eddy currents from being induced in the support structure. The support structure therefore cannot be heated by the eddy currents occurring on the application of strong magnetic fields. In addition, the use of examination processes such as nuclear magnetic resonance cannot result in image artefacts due to shielding effects.

A support structure which is free from support elements encircling the circumference of the casing in a closed manner is obtained, for example, if the support structure is formed by a support helix. In this case, high supporting forces can be applied to the wall of the vessel without the risk of magnetic fields inducing eddy currents in the support structure.

The non-metallic materials include, in particular, materials based on chitin or chitosan. Materials of this type are atoxic, there are no known allergic reactions, and the mechanical properties are variably adjustable. In addition, chitin or chitosan is approved by the American Food and Drug Administration (FDA) as a food additive. Materials of this type are therefore suitable for the connecting elements between the support elements. In addition, polymers which are likewise atoxic, do not cause allergic reactions and are sufficiently resilient also appear to be suitable.

In one embodiment of the support prosthesis, the connecting elements can be in the form of strips and extend in the circumferential direction in such a way that the connecting elements partially cover support elements arranged next to one another. In this case, particularly high connecting strength between the individual elements can be expected.

In a further embodiment, the connecting elements are in the form of strips and extend in the longitudinal direction via a large number of support elements In this case, the requirements placed on the resilience of the connecting elements are particularly low, as the connecting elements have to be stretched only slightly on expansion of the support elements.

In addition, it is possible to arrange the connecting elements in gaps between the support elements. In this case, the connecting elements can be produced by immersing the pre-assembled support elements into a solution containing the material used for the connecting elements. In addition, it is also conceivable to add the solution dropwise or by spraying and to introduce the solution into the gaps between the support elements by capillary action.

### BRIEF DESCRIPTION OF THE DRAWINGS

Further details and advantages of the invention emerge from the following description in which embodiments are described in detail with reference to the appended drawings, in which:

FIG. 1 is a perspective view of a stent;

FIG. 2 is a plan view onto two stent support rings which are arranged next to each other and are connected via a strip-like connecting element;

FIG. 3 is a plan view onto two support rings which are arranged next to each other and can be connected in various ways by strip-like connecting elements extending in the longitudinal direction;

FIG. 4 is a plan view onto support rings arranged next to one another with a connecting element arranged in a gap between the support rings;

FIG. 5 is a plan view onto support rings arranged next to one another with a further connecting element arranged in a gap between the support rings;

FIG. 6 is a plan view onto two adjacent support rings, the gap between which is filled completely by a connecting element;

FIG. 7 is a plan view onto two support rings covered by a connecting element configured at the surface; and

FIG. 8 is a plan view onto a cut-open casing of a stent comprising a support helix.

### DETAILED DESCRIPTION OF THE INVENTION

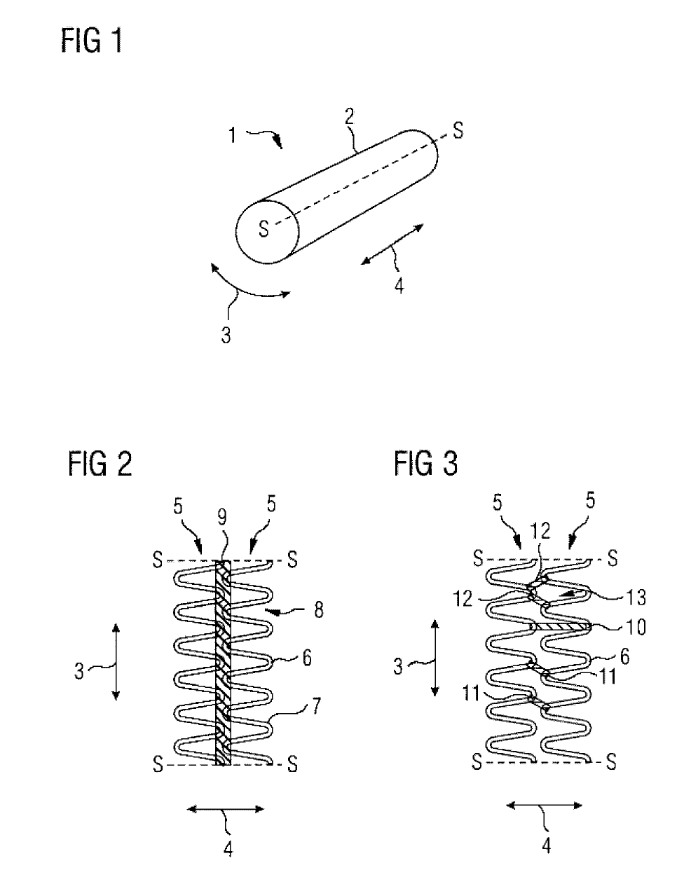

FIG. 1 is a perspective view of a stent 1 comprising a tubular casing 2. The casing 2 has a large number of support rings 5 which extend in a circumferential direction 3, are arranged next to one another in a longitudinal direction 4 and are shown in the following FIG. 2 to 7 on an enlarged scale and cut open along a sectional line S-S.

The support rings 5 have a meandering, in particular an undulating, course. In the case of the support rings 5 shown in FIG. 2 to 7, which display an undulating course, curves 6 are connected by straight support struts 7. A curve 6 and two adjacent support struts 7 form, in each case, a loop 8. Successive loops 8 therefore share a respective support strut 7.

For implantation, the stent 1 is crimped onto what is known as a balloon catheter. The balloon catheter is then brought to the point to be widened of the vessel to be treated, where it is expanded. This stretches the support rings 5 in the circumferential direction 3. In particular, the curves 6 are in the extreme scenario bent about a radial axis sufficiently far for the support ring 5 to extend in a plane. The circumference of the support rings 5 is typically enlarged in this case by approximately 4.5 times.

In the embodiment shown in FIG. 2, the meandering patterns of the support rings 5 arranged next to one another each extend in phase. This means that the loops 8, open toward the left, of a first support ring 5 and loops 8, open toward the left, of a second support ring 5 and also loops 8, open toward the right, of the first support ring 5 and loops 8, open toward the right, of the second support ring 5 oppose one another in each case. In the region of the curves 6, the support rings 5 are connected, in the embodiment shown in FIG. 2, by a connecting strip 9 extending in the circumferential direction 3 between the support rings 5. For the connecting strip 9, use is preferably made of a material which, on account of its resilient properties, is able to follow the expansion of the support rings 5. Otherwise, the support rings 5 will become detached from the connecting strip 9 during the radial expansion. In order for the connecting strip 9 to ensure the distance between the support rings 5 even in this case, the radial thickness of the support ring 5 must be sufficiently great for the support rings 5 to be set apart from the annular connecting strip 9 even in the stretched state.

FIG. 3 shows a further embodiment of the stent 1 in which the support rings 5 are arranged next to one another in phase. FIG. 3 shows various possibilities for connecting the support rings 5 using longitudinally oriented connecting elements. FIG. 3 thus shows a connecting strip 10 which extends in the longitudinal direction 4 and connects the curves 6 of two loops 8 open toward the left. On expansion of the stent 1, the connecting strip 10 hardly needs to be expanded. On expansion of the stent, a connecting strip of the type of the connecting strip 10 is typically extended by up to 1.5 times. There can thus be used for the connecting strip 10 comparatively rigid material which sets the support rings 5 apart from one another. This latter aspect is important if the stent 1 is introduced using a balloon catheter, as otherwise the support rings 5 are pushed onto one another on radial expansion.

In addition, it is possible to connect the curves 6 of adjacent loops 8 by short connecting strips 11. The connecting strips 11 can each be arranged in such a way that a respective curve 6 of the one support ring 5 is connected to a closest curve 6 of the other support ring 5. In this case, the stent is shortened considerably on expansion of the support rings 5 unless predetermined breaking points are provided in the connecting strips 11.

In a modified embodiment of the stent 1, short connecting strips 12 connect a respective curve 6 of the one support ring to two differing curves 6 of the other support ring 5. As, in this embodiment, the connecting strips 12 prevent the opening of a loop 13 of the second support ring 5, the short connecting strips 12 are torn or detached from the support rings 5 on expansion of the support rings 5. There is therefore preferably provided at the connecting strips 12 a predetermined breaking point at which the short connecting strips 12 are torn. After the expansion of the support rings 5, the support rings 5 are no longer connected to one another. The support rings 5 and therefore freely movable in this case, so this embodiment would seem to be particularly suitable for vessels exposed to frequent and periodic deformations. Examples of vessels of this type include the coronary vessels which deform on each movement of the myocardium.

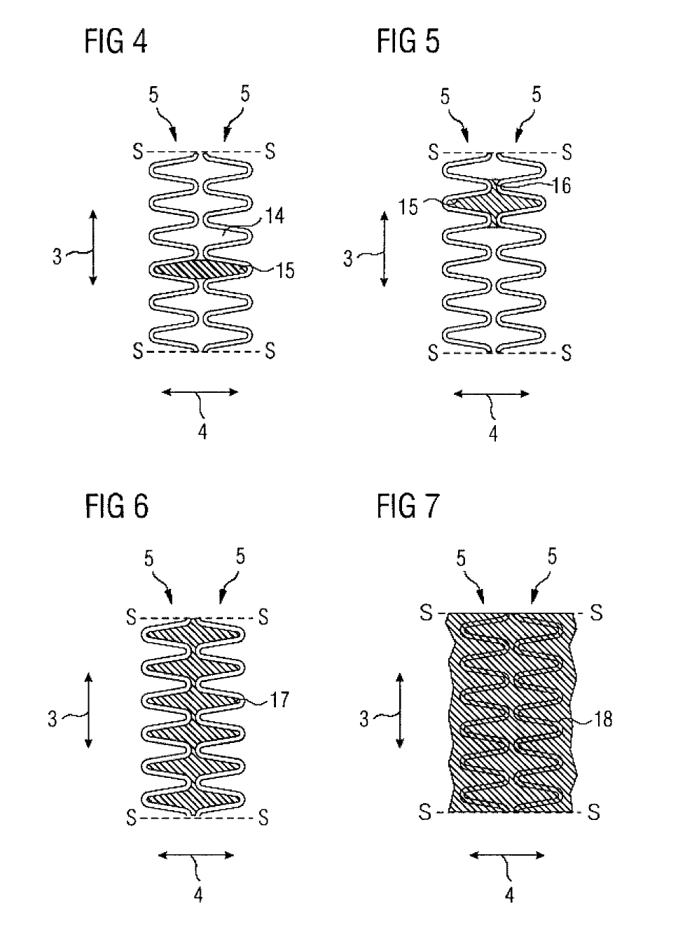

FIG. 4 shows a further embodiment in which the meandering pattern of support rings located next to one another is phase-offset through 180° in each case. As a result, the curves 6 of support rings 5 located next to one another are in each case tightly packed. The loops 8 of support rings located next to one another therefore form chambers 14 in which there can be formed, for example, a connecting layer 15 of the type shown in FIG. 4.

The connecting layer 15 does not necessarily have to be made of an extremely resilient material. For on expansion of the support rings 5, the connecting layer 15 will tear off along the support struts 7. In the region of the curves 6, on the other hand, the connecting layer 15 continues to adhere to the support rings 5. If the connecting layer 15 is sufficiently rigid, the support rings 5 are set apart from one another. The connecting layer can be produced in a particularly simple manner, as the chamber 14 shaped by the support rings 5 located next to one another can be used for the shaping of the connecting layer 15. For the chamber 14 delimits the lateral extension of the connecting layer 15. It is thus, for example, possible to attach the support rings 5 to a base and to fill the chamber 14 with the material of the connecting layer 15.

This can be carried out, for example, by applying a suitable solution dropwise. The solution is in this case, as shown in FIG. 5, drawn by capillary action even into narrowings 16 between curves 6, located close together, of support rings 5 arranged next to one another.

In principle, it is conceivable to fill all of the chambers 14, between the support rings 5, with connecting layers 17 extending over the entire circumference of the support rings 5. The connecting layers 17 can be produced most easily by immersing the casing 2 into a solution of the material used for the connecting layers 17.

Finally, FIG. 7 shows an exemplary embodiment of the stent 1, in which a continuous connecting layer 18 has been attached to the casing 2. This is especially advantageous if the material for the connecting layer 18 has a high viscosity when dissolved and can be rolled onto the casing 2 of the stent 1. It is also conceivable to push and then crimp the material of the connecting layer 18, in the form of a tube, onto the stent 1. In addition, it is also conceivable to form the connecting layer 18 as a tube, onto the outside of which the individual support rings 5 are pushed and then fixed.

It should be noted that the connecting layer 18 can also be reticulate in its construction.

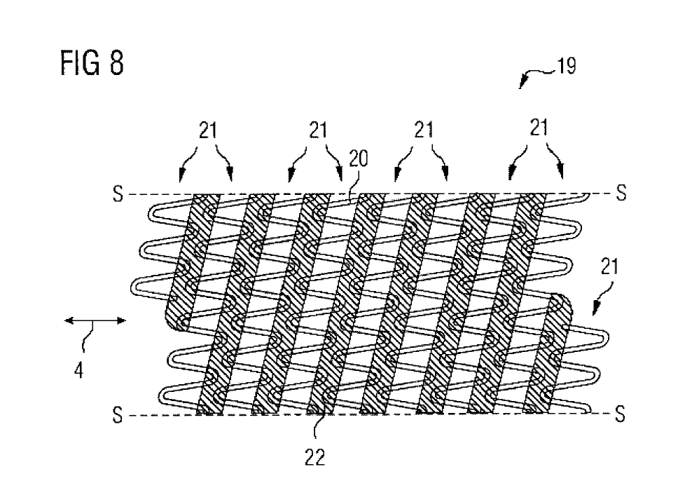

FIG. 8 shows a further stent 19 which is cut open along the sectional line S-S and the supporting structure of which is formed by a metallic support helix 20. The stent 19 is shown such as it would appear cut open along a sectional line S-S and resting flat on a planar surface. In this state, the support helix 20 is divided into helix portions 21 which respectively correspond to the course of the support helix 20 when revolved through 360°. The helix portions 21 display a meandering course and extend transversely to the longitudinal direction 4 of the stent 19.

The individual helix portions 21 can then be connected along the longitudinal direction 4 by connecting elements of the type of the connecting strips to 9 to 12 and also the connecting surfaces 15, 17 and 18. In the stent 19 shown in FIG. 8, the helix portions 21 are connected, for example, by a connecting strip 22 of the type of the connecting strip 9.

In addition to its high resilience, the stent 19 has the further advantage that no extensive eddy currents can be induced in the support helix 20. The stent 19 therefore does not obstruct the application of therapy and diagnosis processes which operate with strong magnetic fields. For the stent 19 cannot be warmed or heated, in the event of strong magnetic fields, by the occurrence of eddy currents. Nuclear magnetic resonance also does not lead to the generation of image artefacts due to shielding effects. On the contrary, it should even be possible to obtain images of the interior of the stent 19 using the aforementioned processes. However, this presupposes a sufficiently large distance between the helix portions 21.

It is also conceivable to position the stent 19 using catheters having a magnetic tip which is navigated with the aid of strong magnetic fields.

The stents 1 and 19 described in the present text are particularly suitable for the widening of coronary vessels. As they have a high degree of flexibility, they can also be introduced into markedly curved vessels. The stents 1 and 19 are also able to follow frequent periodic movements of the vessels.

Possible materials for the connecting elements 9 to 11 and also 15, 17, 18 and 22 include materials produced based on chitin. Chitin, which is N-acetyl-D-glucose-2-amine, can also be mixed with sclerotine or the precursors thereof. The acetyl groups of chitin can be separated by boiling, thus producing chitosan. It would seem conceivable to produce the connecting elements 9 to 11 and also 15, 17, 18 and 22 also based on chitosan.

Further possible materials for the connecting elements 9 to 11 and also 15, 17 and 18 include other materials containing glucose amines or derived therefrom such as, for example, cellulose derivatives.

Finally, it is also conceivable to use for the connecting elements 9 to 11 and also 15, 17, 18 and 22 plastics materials from the group of the polymers or elastomers, provided that they are biocompatible.

It should be noted that the materials used for the connecting elements 9 to 11 and also 15, 17, 18 and 22 can also contain metallic components. The metallic components can be individual particles, traces or thin coatings which do not provide any mechanical connection between the support rings 5 or the helix portions 21.

1. A support prosthesis for vessels or intracorporeal lumens with a tubular casing which can expand in the radial direction and has a metallic support structure with support elements extending transversely to the longitudinal direction, wherein support elements arranged next to one another in the longitudinal direction are connected to the tubular casing by at least one connecting element which extends in the longitudinal direction and is made of a resilient, non-metallic material, and wherein the connecting element is a connecting layer configured in a chamber formed by support elements located next to each other and limiting the lateral extension of the connecting element in the circumferential direction.

2. The support prosthesis according claim 1, wherein the connecting element allows the support elements to stretch in the circumferential direction and sets the support element apart from one another on radial expansion.

3. The support prosthesis according to claim 1, wherein the support elements have curves which can stretch about a radial axis on radial expansion and the connecting elements are attached in the region of the curves.

4. The support prosthesis according to claim 3, wherein the connecting element is attached to the support structure so as to be detachable from the region surrounding the curves on radial expansion.

5. The support prosthesis according to claim 1, wherein the support structure has support rings which are arranged next to one another in the longitudinal direction and are connected to the tubular casing by at least one connecting element made of a resilient, non-metallic material.

6. The support prosthesis according to claim 1, wherein the support structure is free from support elements which encircle the casing in a closed manner in the circumferential direction.

7. The support prosthesis according to claim 6, wherein the support structure is formed by a support helix, of which the helix portions located next to one another in the longitudinal direction are connected to the tubular casing by at least one connecting element made of a resilient, non-metallic material.

8. The support prosthesis according to claim 1, wherein the support elements extend in meandering form.

9. The support prosthesis according to claim 1, wherein the non-metallic material is produced based on chitin.

10. The support prosthesis according to claim 9, wherein the non-metallic material is produced based on chitosan.

11. The support prosthesis according to claim 1, wherein the material is a polymer.

12. The support prosthesis according to claim 1, wherein the connecting element is a connecting layer extending in narrowings between support elements located next to each other.

13. The support prosthesis according to claim 2 wherein the connecting element is a connecting layer extending in narrowings between support elements located next to each other.

14. The support prosthesis according to claim 3 wherein the connecting element is a connecting layer extending in narrowings between support elements located next to each other.

15. The support prosthesis according to claim 4 wherein the connecting element is a connecting layer extending in narrowings between support elements located next to each other.
